(19) United States
(12) Patent Application Publication
(10) Pub. No.: US 2018/0325207 A1
(43) Pub. Date: Nov. 15, 2018

(54) Weight And Activity Monitoring Footwear

(71) Applicant: Verily Life Sciences LLC, Mountain View, CA (US)

(72) Inventor: Benjamin David Krasnow, Redwood City, CA (US)

(73) Assignee: Verily Life Sciences LLC, Mountain View, CA (US)

(21) Appl. No.: 15/974,780

(22) Filed: May 9, 2018

Related U.S. Application Data
(60) Provisional application No. 62/503,396, filed on May 9, 2017.

(51) Int. Cl. A43B 3/00; A61B 5/00; A63B 24/00

(52) U.S. Cl. CPC A43B 3/0005; A61B 5/1038; A63B 24/0062; A61B 5/6807

(57) ABSTRACT

The technology described herein relates to weight and activity monitoring footwear. In an implementation, an article of footwear is disclosed. The article of footwear includes a sole, a plurality of pressure sensors, a motion sensor, and a microcontroller. The sole has a plurality of pressure chambers. The plurality of pressure sensors take pressure readings from the plurality of pressure chambers. The motion sensor senses motion of the article of footwear. The microcontroller is operatively coupled with the plurality of pressure sensors and the motion sensor and is configured to detect when a subject enters a standing state from the motion of the article of footwear, and in response to detecting the standing state, determine a weight applied by the subject against the sole from the pressure readings. The plurality of pressure chambers are disposed to support the weight applied by the subject against the sole in the standing state.

### BACKGROUND

Adverse health conditions such as obesity have negative impact on depression, heart disease, and type-II diabetes. These health conditions currently account for a sizeable portion of healthcare costs in the United States and across the globe. Exercise and other health-related activities and programs can counteract many of the ill effects of these conditions. Monitoring these activities has been shown to aid in the longevity and success of many exercise and health-related programs.

Unfortunately, monitoring exercise and health-related activities can be disjointed, inaccurate and burdensome. For example, belt- and wrist-mounted fitness tracking devices currently exist but can, and often are, left at home. Regardless, these fitness tracking devices cannot accurately measure the body weight of a user. Shoe devices with multi-region force sensors are able to show areas of relative force concentration beneath a foot of a user, e.g., to produce heatmap images. However, these devices still cannot accurately measure the body weight of a user.

### SUMMARY

Examples discussed herein relate to weight and activity monitoring footwear. In an implementation, an article of footwear is disclosed. The article of footwear includes a sole, a plurality of pressure sensors, a motion sensor, and a microcontroller. The sole has a plurality of pressure chambers. The plurality of pressure sensors take pressure readings from the plurality of pressure chambers. The motion sensor senses motion of the article of footwear. The microcontroller is operatively coupled with the plurality of pressure sensors and the motion sensor and is configured to detect when a subject enters a standing state from the motion of the article of footwear, and in response to detecting the standing state, determine a weight applied by the subject against the sole from the pressure readings. The plurality of pressure chambers are disposed to support the weight applied by the subject against the sole in the standing state.

This Overview is provided to introduce a selection of concepts in a simplified form that are further described below in the Technical Disclosure. It may be understood that this Overview is not intended to identify key features or essential features of the claimed subject matter, nor is it intended to be used to limit the scope of the claimed subject matter.

### BRIEF DESCRIPTION OF THE DRAWINGS

In order to describe the manner in which the above-recited and other advantages and features can be obtained, a more particular description is set forth and will be rendered by reference to specific examples thereof which are illustrated in the appended drawings. Understanding that these drawings depict only typical examples and are not therefore to be considered limiting of its scope, implementations will be described and explained with additional specificity and detail through the use of the accompanying drawings.

FIG. 1 depicts an article of footwear for monitoring weight and activity of a subject (or wearer of the footwear), according to some embodiments.

FIG. 2 depicts example weight and activity monitoring components of an article of footwear, according to some embodiments.

FIG. 3 depicts an example operational architecture including weight and activity monitoring footwear, according to some embodiments.

FIG. 4 depicts a flow diagram illustrating example operations for weight and activity monitoring, according to some embodiments.

FIG. 5 depicts a flow diagram illustrating example operations for weight and activity monitoring, according to some embodiments.

FIG. 6 depicts a flow diagram illustrating example operations for determining a weight applied by a subject against a sole of the article of footwear based on pressure readings from pressure sensors when a subject is in a ‘standing state,’ according to some embodiments.

FIG. 7 depicts a side-view of an article of footwear for monitoring weight and activity of a subject (or wearer of the footwear), according to some embodiments.

FIG. 8 depicts a top view of an article of footwear having a grouped pressure sensor with multiple sensing elements for monitoring weight and activity of a subject (or wearer of the footwear), according to some embodiments.

FIG. 9 depicts a block diagram illustrating a computing system suitable for implementing the electronic calendar sharing technology disclosed herein, including any of the applications, architectures, elements, processes, and operational scenarios and sequences illustrated in the Figures and discussed below in the Technical Disclosure.

The drawings have not necessarily been drawn to scale. Similarly, some components and/or operations may be separated into different blocks or combined into a single block for the purposes of discussion of some of the embodiments of the present technology. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

Examples are discussed in detail below. While specific implementations are discussed, it should be understood that this is done for illustration purposes only. A person skilled in the relevant art will recognize that other components and configurations may be used without parting from the spirit and scope of the subject matter of this disclosure. The implementations may include machine-implemented methods, computing devices, or computer readable medium.

The technology described herein is directed to weight and activity monitoring footwear and, more particularly, to weight and activity monitoring footwear having weight and activity monitoring components that facilitate precise body weight measurements, e.g., on the order of 0.1 lbs. The weight and activity monitoring components include multiple controlled pressure chambers, multiple pressure sensors that sense pressure readings of the pressure chambers based on movement of the footwear, and machine-learning algorithms that convert raw sensor pressure readings into stable weight measurements.

The controlled pressure chambers are disposed at various regions in the soles of the footwear for fully supporting the body weight applied by a subject or wearer of the footwear when the subject is in a ‘standing state.’ Among other benefits, the weight and activity monitoring footwear continuously monitors a subject and automatically measures the body weight of a subject when accurate readings can be taken, e.g., when the subject is in a ‘standing state.’ The readings can be taken without requiring the subject to step on a scale or manually record the readings. Additionally, the precise readings allow the weight and activity monitoring footwear to provide a continuous, unbroken graph of body weight over time. This allows a subject to, for example, monitor how much weight has been lost through sweating during exercise, or gained after a meal or through hydration.

In some embodiments, the weight and activity monitoring footwear detects at least four regions of force beneath the foot, and can provide information about, among other information, pronation, heel-strike, forefoot running or walking, and Left/Right balance. The weight and activity monitoring footwear can also provide an accurate “steps counter” which cannot be easily forgotten at home unlike belt- or wrist-mounted fitness tracking devices.

The footwear discussed herein is primarily depicted and discussed with reference to shoes. It is appreciated that footwear may be any outer covering for feet, such as shoes, boots, or sandals. Additionally, footwear as discussed herein generally includes a pair of articles, e.g., a shoe for each foot of the subject (or wearer) of the footwear.

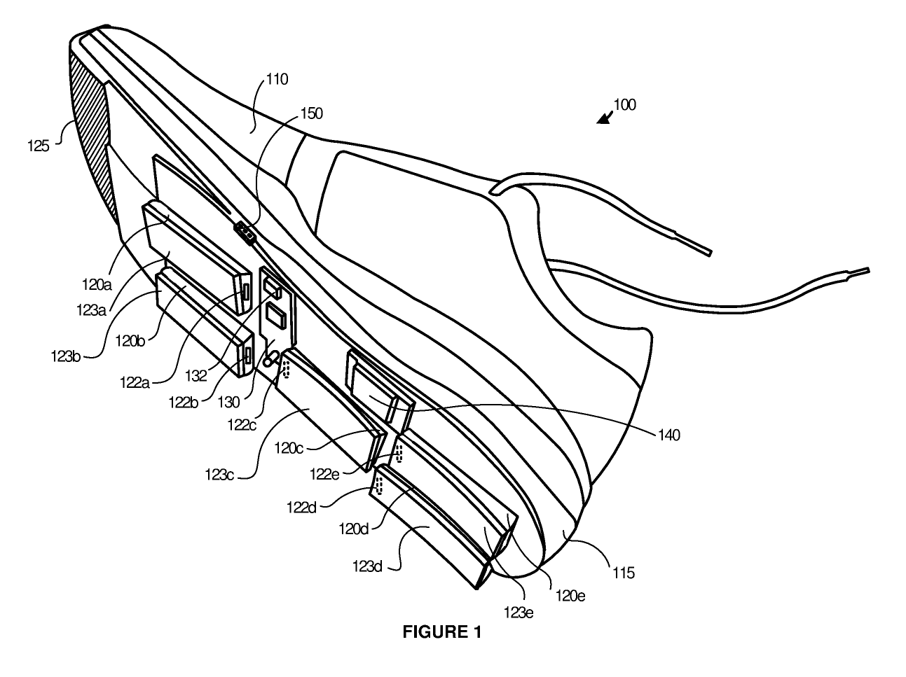

FIG. 1 depicts an article of footwear 100 for monitoring weight and activity of a subject (or wearer of the footwear), according to some embodiments. The article of footwear 100 includes an upper 110 and a sole 115. The sole 115 includes a tread 125 that is disposed along a contact surface with the ground. As shown in the example of FIG. 1, the tread 125 is partially removed along with a portion of the sole 115 to reveal weight and activity monitoring components molded or embedded in the sole 115. The weight and activity monitoring components include multiple pressure chambers 120a-120e each having a corresponding pressure sensor 122a-122e, a microcontroller 130, a power source 140, and a communication module 150. Additional or fewer components are possible.

The microcontroller 130 can include a motion (or movement) sensor 132. In some embodiments, the motion sensor 132 may not be co-located with microcontroller 130. The motion sensor 132 can be, for example, a three-axis digital accelerometer that provides a digital output indicating acceleration of the article of footwear 100 to the microcontroller 130.

Additional sensors, such as ambient sensors (not shown), are also possible. For example, in some embodiments, the microcontroller 130 can correct for temperature and barometric pressure differences by sampling hydraulic pressures, e.g., when a subject's foot is mid-stride and not applying any normal force to the shoe.

One or more the weight and activity monitoring components can be molded or embedded in sole 115. However, in some embodiments, at least some of the weight and activity monitoring components may be located elsewhere on the article of footwear 100, e.g., on or within upper 110. Additionally, it is appreciated that footwear includes a pair of articles each of which have weight and activity monitoring components that can be shared or redundant.

The pressure sensor 122a-122e are adapted to take pressure readings from corresponding pressure chambers 120a-120e. The pressure chambers 120a-120e can be hydraulic chambers, pneumatic chambers, or some other liquid or gas filled chambers, (including combinations or variations thereof) molded into at least four quadrants of the sole 115. Likewise, pressure readings can be hydraulic pressure readings, pneumatic pressure readings, combinations thereof, etc.

The pressure sensors 122a-122e can be electronic sensors of various types. In some embodiments, one or more of the pressure sensors 122a-122e can be silicon chips that have a thinned membrane and detect the strain of the membrane with microfabricated strain gauges. These sensors can be very small, e.g., on the order of 1×1 mm, and can provide digital outputs of the pressure readings.

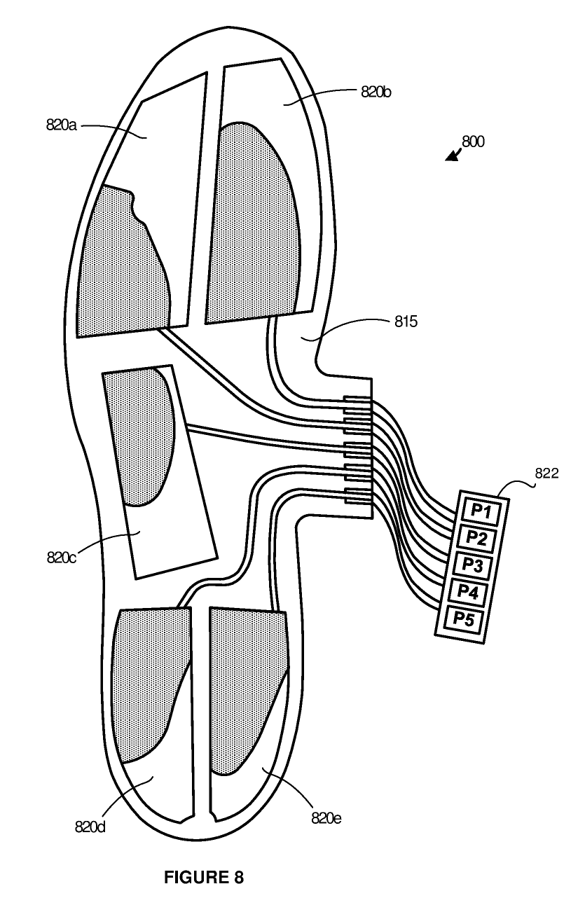

In some embodiments, multiple of pressure sensors 122a-122e can be grouped, e.g., placed or fabricated, on a single silicon die. Grouped sensors can provide a smaller overall footprint for space and cost optimizations. With grouped sensors, the pressure in each chamber can be routed through molded passageways to a single location where the grouped sensor can take the pressure reading by sensing or measuring the pressure in the pressure chambers. An example depicting a grouped sensor is shown and discussed in greater detail with reference to FIG. 8.

In some embodiments, the pressure chambers 120a-120e are shaped like “bellows” allowing a pressure chamber to decrease in height without significant force. The “bellows” ensure that the force applied, e.g., by a foot of the wearer of the shoe, to the pressure chambers 120a-120e is entirely supported by the pressure chambers 120a-120e themselves, e.g., fluid or pneumatic pressure of the pressure chambers 120a-120e.

Tension members 123a-123e can be used to connect or attach the pressure chambers 120a-120e to the tread 125 at one or more locations. As shown in the example of FIG. 1, each pressure chamber 120a-120e includes a respective tension member 123a-123e. However, in some embodiments, tension members 123a-123e may only be used at particular sections or regions, e.g., at heel and ball areas, to handle the higher shear forces encountered by the article of footwear 100 at those regions. The tension members 123a-123e are flexible, not significantly influencing normal force, but are taut when a shear force is applied. Like other weight and activity monitoring components, the tension members 123a-123e can be molded or embedded in the sole 115. The tension members 123a-123e can be fabricated from rubber-coated fiberglass or some other strong flexible material.

The microcontroller 130 in the article of footwear 100 and the microcontroller in an opposing article of footwear (not shown), e.g., opposing shoe, use output from the motion sensor 132 to detect when a wearer of the article of footwear 100 is in a “standing state,” e.g., the footwear is flat on the ground and relatively still. It is appreciated that a typical subject, e.g., a human, when standing, has slight continuous weight shift between feet and within each foot. These small movements (or weight shifts) are detected by microcontroller 130 and used to estimate when the subject is standing ‘normally’ or in a state from which accurate weight measurements can be taken. Additionally, the microcontroller 130 can determine when the subject is moving and, more particularly, when particular movements are made by the subject. These movement can be used to trigger other activity monitoring functionality.

In response to detecting that the subject is in a “standing state,” the microcontroller 130 determines a weight applied by the subject against the sole 115 of the article of footwear 100. The weight applied by the subject against the sole 115 of the article of footwear 100 can be determined by sampling pressure reading from the plurality pressure sensors 122a-122d during the “stating state,” converting the pressure readings to weight measurements and summing the weight measurements.

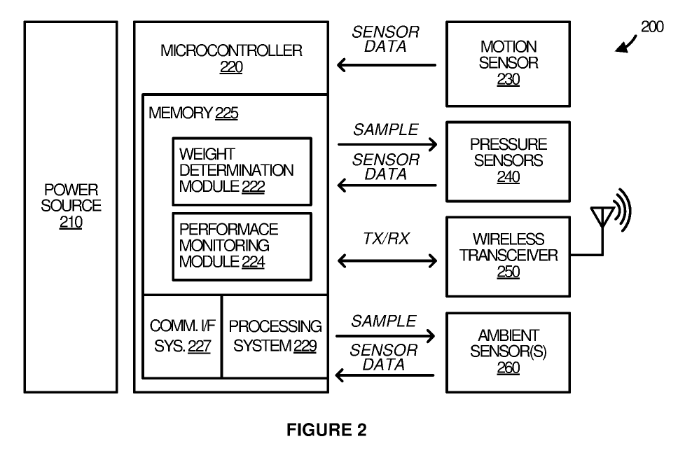

FIG. 2 depicts example weight and activity monitoring components 200 of an article of footwear, according to some embodiments. The article of footwear can be the article of footwear 100 of FIG. 1, although alternative configurations are possible. As illustrated in the example of FIG. 2, the weight and activity monitoring components 200 include a power source 210, a microcontroller 220, a motion sensor 230, pressure sensors 240, a wireless transceiver 250, and one or more ambient sensors 260. Additional or fewer components are possible.

The power source 210 provides power to activity monitoring components 200. Any energy storage device may be employed. For example, the power source 210 can include one or more batteries and related charging and regulator circuitry. In some embodiments, one or more batteries may be used for the life of the product or may be periodically removed and replaced. Alternately, power source 210 can include a rechargeable power source, e.g., a lithium-ion battery, in which a direct current (DC) energy source is periodically plugged into a receptacle of the article of footwear, e.g., universal serial bus (USB). In some embodiments, the power source 210 may also include a kinetic energy source which generates power through movement of the shoe.

The microcontroller 220 can be a small computer or other circuitry that retrieves and executes software from memory 225. The microcontroller 220 may be implemented within a signal device or system on a chip (SoC) or may be distributed across multiple processing devices that cooperate in executing program instructions. As shown in the example of FIG. 2, the microcontroller 220 includes memory 225, a communication interface 227, and a processing system 229. The microcontroller 220 is operatively or communicatively coupled with various sensors including the motion sensor 230, the pressure sensors 240, and the one or more ambient sensors 260. Additionally, the microcontroller 220 is operatively or communicatively coupled with the wireless transceiver 250.

The memory 225 can include program memory and data memory. As shown, memory 225 includes a weight determination module 222 and a performance monitoring module 224. Other modules are also possible. Although shown as software modules in the example of FIG. 2, functionality of the weight determination module 222 and the performance monitoring module can be implemented individually or in any combination thereof, partially or wholly, in hardware, software, or a combination of hardware and software.

The communication interface 227 may include communication connections and devices that together facilitate communication with auxiliary devices such as, for example, smartphones or watches, as well as other articles of footwear via at least wireless transceiver 250. The processing system 229 can include one or more processor cores that are configured to retrieve and execute the weight determination module 222 and the performance monitoring module 224 for performing various weight and activity monitoring functions as discussed herein.

The motion sensor 230 senses motion of the article of footwear. The motion sensor can be, for example, a three-axis digital accelerometer that provides a digital output indicating acceleration of the article of footwear to the microcontroller 130.

The pressure sensors 240 take pressure readings from pressure chambers disposed in the sole of the article of footwear. The pressure sensors 240 can be can be electronic sensors of various types that take pressure readings and provide digital samples indicating raw values of the readings to microcontroller 220. For example, the pressure sensors 240 can be silicon chips that having thinned membranes and an apparatus capable of detecting strain of the membrane with microfabricated strain gauges. As discussed above, the sensors can be very small, e.g., on the order of 1×1 mm, and can provide digital outputs of the pressure readings.

The wireless transceiver 250 can be for example a Bluetooth or Bluetooth Low Energy (BLE) transceiver. An infrared transceiver is also possible. The one or more ambient sensors 260 can include, among other ambient sensors, temperature and barometric pressure sensors that can be used to correct for different temperature and barometric pressure differences when calculating a weight of a subject.

Referring to the weight determination module 222, in operation, the module can direct the microcontroller to take pressure readings throughout the day when a subject is in a “standing state.” The pressure readings can be fed into a machine-learning algorithm to determine precise body weight of the subject. For example, the subject might be a human standing still while holding a coffee mug. The mug adds a little bit of extraneous weight to the measurement. Accordingly, the machine-learning algorithm can average and filter the measurements taken over the day to calculate a precise body weight of the subject and monitor the weight of the subject over time.

Raw pressure readings from the pressure sensors are converted into weights using a conversion algorithm that can be factory calibrated. For example, each pressure chamber can deform slightly when loaded and thus its cross-sectional area enlarges as more load is applied. This causes the relationship between force and pressure to be slightly non-linear. This behavior can be approximated for each pressure chamber with a second order polynomial.

The coefficients of the second order polynomial for each pressure chamber can be determined with an iterative solver program. The solver uses the pressure readings from each pressure sensor associated with each pressure chamber, in addition to the total load, to determine separate polynomial coefficients for each pressure chamber. Once calibrated, the microcontroller 220 uses the coefficients to convert raw pressure readings from the pressure sensors to corresponding weight measurements.

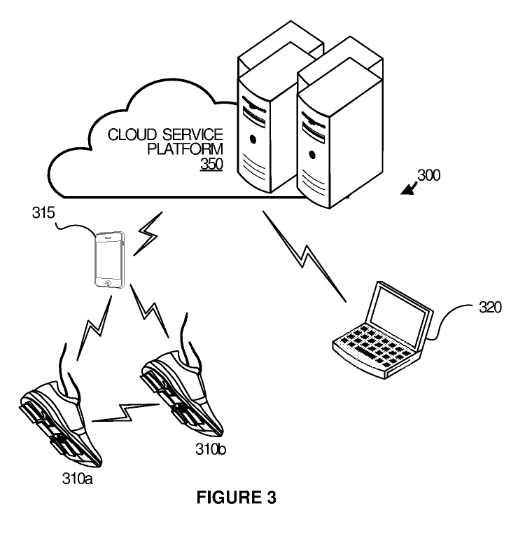

FIG. 3 depicts an example operational architecture 300 including weight and activity monitoring footwear 310, according to some embodiments. The example operational architecture 300 includes the footwear 310, an auxiliary device 315, a cloud service 350, and an access system 320. As shown in the example of FIG. 3, the monitoring footwear 310 includes shoes 310a and 310b. Each shoe 310a and 310b can include weight and activity monitoring components. Some of the components can be shared or redundant.

In some embodiments, the first shoe 310a and the second shoe 310b communicate movement information such as, for example, weight measurements and states of motion, e.g., whether they are in a ‘standing state.’ For example, weight measurements from each shoe 310a and 310b can be synchronized by sending the values via radio frequency (RF), infrared (IR) beaconing between shoes, or the like.

Additionally, weight information can be communicated between the shoes. For example, if shoe 310a detects a ‘standing state’ then shoe 310a can request and wait for a measurement from 310b. If shoe 310b also detects ‘standing state’ behavior (at the exact same time as determined by time markers), then a total weight measurement of a subject can be made that includes the weight of the user attributed to both shoe 310a and shoe 310b.

The measurements from each shoe 310a and 310b may be stored and transferred to auxiliary device 315 (or, alternatively, transferred directly to the cloud). In some embodiments, the shoes 310a and 310b may not communicate, or may only communicate movement information. In such instances, total weight determinations may subsequently be made by the auxiliary device 315 with can receive data including weight, time and movement information via a wireless connection. For example, the auxiliary device 315 can match timestamps included with weight measurements and use the timestamps to determine a total weight at a particular time given that both shoes were in a ‘standing state’ at the time.

Processing of weight data and other pressure readings can include various data filtering and averaging algorithms. The data filtering and averaging algorithms can be performed, in whole or in part, by one or both microcontrollers in shoes 310a and 310b, by the auxiliary device 315, or by the cloud service (cloud service platform 350).

The auxiliary device 315 can include a computing system or collection of systems having a suitable computing architecture for carrying out the various functions discussed herein including processing, manipulating and rendering data received from the shoes 310a and 310b and cloud service platform 350, of which computing system 900 is representative.

Cloud service platform 350 is representative of any cloud service or collection of services that is configured to facilitate various processing and manipulating of data received from the shoes 310a and 310b, as well as other shoes (not shown) that are communicatively coupled to the service. Additionally, the cloud service platform 350 can provide information to access systems such, as for example, access system 320 for rendering monitoring information to subjects, physicians, etc., on various devices.

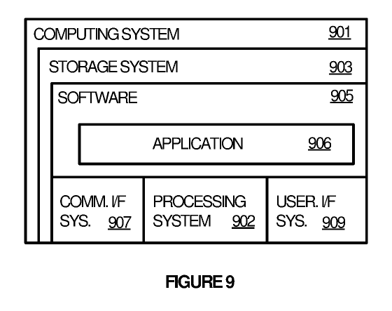

The cloud service platform 350 may include server computers, blade servers, rack servers, and any other type of computing system (or collection thereof) suitable for carrying out a service or collection of services and for interfacing with the users of the service. The cloud service platform 350 can include GUIs (graphical user interface) running on a PC, mobile phone device, a Web server, or even other application servers. Such systems may employ one or more virtual machines, containers, or any other type of virtual computing resource in the context of supporting a service or collection of services, e.g., an analytics platform, of which the computing system 901 of FIG. 9 is representative.

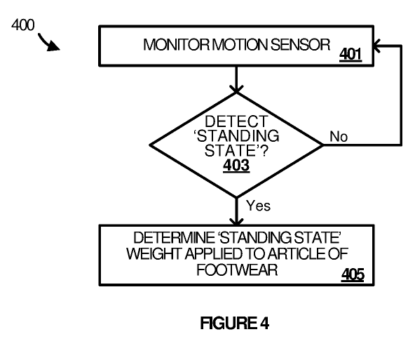

FIG. 4 depicts a flow diagram illustrating example operations 400 for weight and activity monitoring, according to some embodiments. More specifically, the example of FIG. 4 depicts operations of an article of footwear for making a determination of weight applied by a subject against a sole of the article of footwear. The example operations 400 may be performed in various embodiments by an article of footwear such as, for example, article of footwear 100 of FIG. 1, or one or more microcontrollers, modules, engines, or components associated therewith.

To begin, at 401, the article of footwear monitors a motion sensor. In some embodiments, the sensor data may be periodically monitored or sampled while in a low power state. A low power mode or passive circuit can be used to monitor the motion sensor.

At decision 403, the article of footwear determines when a subject enters a ‘standing state.’ In some embodiments, a ‘standing state’ can be indicative of a time when an accelerometer reports acceleration values in the Z direction between −0.97 and −1.02 g. These limits can be used to determine that the subject is on level ground and not moving. Other methods for detecting a ‘standing state’ are also possible. In some embodiments, machine learning algorithms can aggregate standing behavior of many users (in the cloud) and refine what constitutes a ‘standing state’ over time. Additionally, in some instances, individual tendencies of subjects can be used to create personalized definitions of a ‘standing state.’

If a ‘standing state’ is not detected, then the article of footwear continues to monitor the motion sensor. Otherwise, when the ‘standing state’ is detected, at 405, the article of footwear determines a weight applied by the subject against the sole based on pressure readings. As discussed herein, it is assumed that the weight of the subject is fully or completely supported by the pressure chambers during the ‘standing state’ to achieve the most accurate measurements. During a ‘standing state,’ many weight measurements may be taken. For example, the ‘standing state’ may trigger sampling of the pressure sensors. In some embodiments, the sampling rate of the pressure sensors can be between 0.25-3 milliseconds with a weight determination being made for each sample. Alternative configurations with different or variable sampling rates are possible.

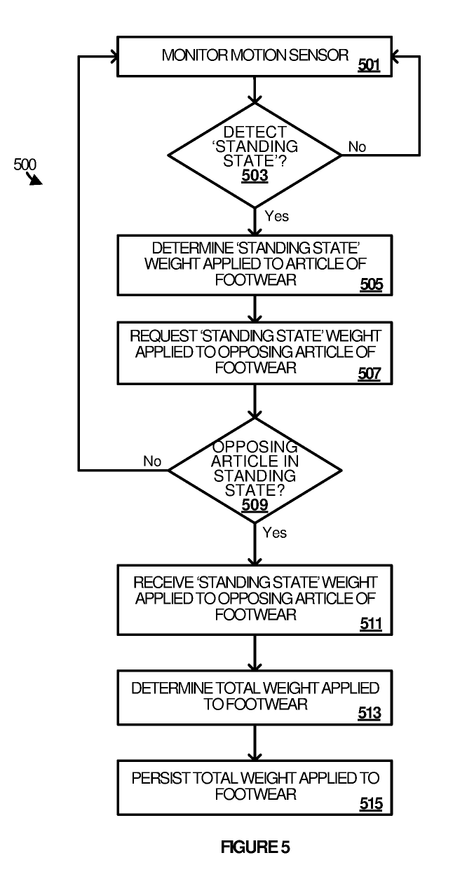

FIG. 5 depicts a flow diagram illustrating example operations 500 for weight and activity monitoring, according to some embodiments. More specifically, the example of FIG. 5 depicts operations of an article of footwear for making a determination of total weight applied by a subject against footwear, e.g., an article of footwear and opposing article of footwear. The example operations 500 may be performed in various embodiments by an article of footwear such as, for example, article of footwear 100 of FIG. 1, or one or more microcontrollers, modules, engines, or components associated therewith.

Operations 501, 503 and 505 are similar to operations 401, 403, and 405 of FIG. 4, and thus are not discussed again here.

At 507, the article of footwear requests a weight measurement applied by the subject against the sole of the opposing article of footwear. At decision 509, the article of footwear receives an indication as to whether the opposing article of footwear is also in a ‘standing state.’ If the opposing article of footwear is not in a ‘standing state,’ then the article of footwear continues to monitor its own motion sensor at step 501.

Otherwise, at 511, the article of footwear waits and receives the weight measurement applied by the subject against the sole of the opposing article of footwear from the opposing article of footwear. At 513, the article of footwear determines a total weight of the subject by summing the weight measurement applied by the subject against the sole of the article of footwear and the weight measurement applied by the subject against the sole of the opposing article of footwear.

Lastly, at 515, the article of footwear persists the total weight. As discussed herein, this total weight can be processed, averaged, and otherwise manipulated by the article of footwear. One or more of the total weight determinations may be sent to an auxiliary device or the cloud.

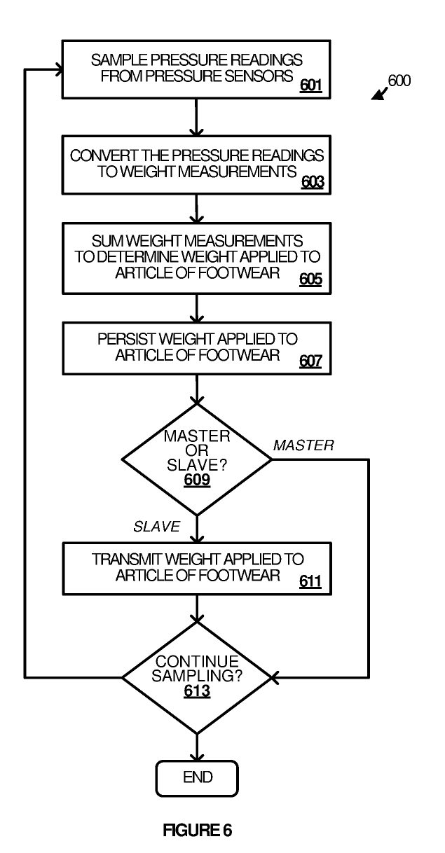

FIG. 6 depicts a flow diagram illustrating example operations 600 for determining a weight applied by a subject against a sole based on pressure readings from pressure sensors when a subject is in a ‘standing state,’ according to some embodiments. More specifically, the example operations 600 illustrate be a detailed representation of operation 505 of FIG. 5, although alternative configurations are possible. The example operations 600 may be performed in various embodiments by an article of footwear such as, for example, article of footwear 100 of FIG. 1, or one or more microcontrollers, modules, engines, or components associated therewith.

Initially, the article of footwear detects a ‘standing state’ based on a motion sensor in the article of footwear. At 601, the article of footwear samples pressure reading from the pressure sensors to obtain pressure readings for each of the multiple pressure chambers of the article of footwear.

At 603, the article of footwear converts the pressure readings for each of the multiple pressure chambers to corresponding weight measurements. As discussed herein, raw pressure readings from the pressure sensors are converted into weights using a quadratic equation. The coefficients for the quadradic equation are predetermined, e.g., at the factory, for each pressure chamber. For example, a raw pressure value of a first pressure sensor measuring a pressure of a first chamber can be represented as:

Raw_{chamber}_{_}₁=a_{chamber}_{_}₁x+b_{chamber}_{_}₁x+c_{chamber}_{_}₁

where a₁, b₁ and c are the predetermined coefficients. The weight measurement corresponding to the raw pressure value of the first chamber can be determined by solving for x.

At 605, the article of footwear sums the weight measurements to determine a weight applied by the subject against the sole of the article of footwear (or a weight applied by the subject on a first article of footwear). At 607, the article of footwear persists the weight applied by the subject against the sole of the article of footwear in memory.

At decision 609, the article of footwear optionally determines whether it is a master or a slave. In some embodiments, either article of a pair of articles can be a master or a slave. In other embodiments, the master may be fixed, e.g., one shoe is always the master, or the master may be the article of footwear that first detects a ‘standing state’ and requests a measurement from the opposing (or slave) article of footwear.

At 611, if the article of footwear is the slave, the article of footwear sends the weight applied by the subject against the sole of the article to the master article of footwear for determination of a total weight of the subject. Alternatively, if the article of footwear is the master, the flow continues to decision 613. At decision 613, the article of footwear determines whether to continue sampling pressure reading from the pressure sensors. In some embodiments, identification of a ‘standing state’ can trigger sampling for a threshold period, e.g., 1-3 seconds.

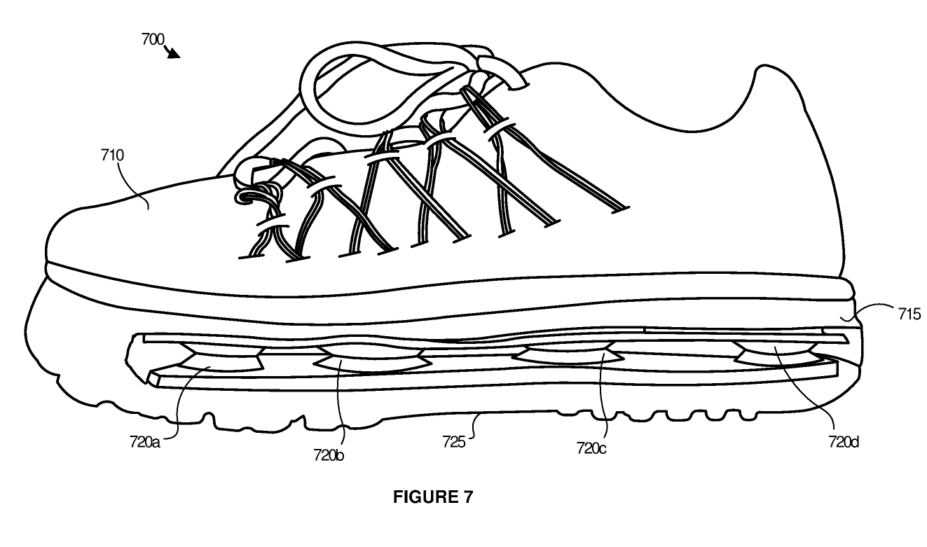

FIG. 7 depicts a side-view of an article of footwear 700 for monitoring weight and activity of a subject (or wearer of the footwear), according to some embodiments. The article of footwear 700 includes an upper 710 and a sole 715. The sole 715 includes a tread 725 that is disposed along a contact surface with the ground. As shown in the example of FIG. 1, a mid-portion of the sole 715 is removed to reveal multiple pressure chambers 720a-720d that are molded or embedded in the sole 715.

The article of footwear is similar to the article of footwear 100 of FIG. 1, however, the multiple pressure chambers 720a-720d are depicted as accordion-style pressure chambers. As discussed above, the pressure chambers can be hydraulic chambers, pneumatic chambers, or some other liquid or gas filled chambers, including combinations or variations thereof, molded into at least four quadrants of the sole 715 to fully support the weight applied by a subject against the sole at least when the subject is in a ‘standing state.’ Other types of pressure chambers are also possible.

FIG. 8 depicts a top view of an article of footwear 800 having a grouped pressure sensor with multiple sensing elements for monitoring weight and activity of a subject (or wearer of the footwear), according to some embodiments.

As shown in the example of FIG. 8, the article of footwear 800 has an upper and a top portion of sole 815 removed to reveal multiple pressure chambers 820a-820e and a grouped sensor 822 including sensor elements P1-P5. The sensor elements can be placed or fabricated, on a single silicon die to provide a small overall footprint for space and cost optimizations. As shown, the pressure in each pressure chamber 820a-820e can be routed through molded passageways to the grouped sensor 822 where each sensor elements P1-P5 takes a pressure reading by sensing or measuring the pressure in the pressure chamber.

FIG. 9 illustrates computing system 901, which is representative of any system or collection of systems in which the various applications, services, scenarios, and processes disclosed herein may be implemented. For example, computing system 901 may include server computers, blade servers, rack servers, and any other type of computing system (or collection thereof) suitable for carrying out the operations described herein. Such systems may employ one or more virtual machines, containers, or any other type of virtual computing resource in the context of supporting enhanced group collaboration.

Computing system 901 may be implemented as a single apparatus, system, or device or may be implemented in a distributed manner as multiple apparatuses, systems, or devices. Computing system 901 includes, but is not limited to, processing system 902, storage system 903, software 905, communication interface system 907, and user interface system 909. Processing system 902 is operatively coupled with storage system 903, communication interface system 907, and an optional user interface system 909.

Processing system 902 loads and executes software 905 from storage system 903. When executed by processing system 902 for deployment of scope-based certificates in multi-tenant cloud-based content and collaboration environments, software 905 directs processing system 902 to operate as described herein for at least the various processes, operational scenarios, and sequences discussed in the foregoing implementations. Computing system 901 may optionally include additional devices, features, or functionality not discussed for purposes of brevity.

Referring still to FIG. 9, processing system 902 may comprise a micro-processor and other circuitry that retrieves and executes software 905 from storage system 903. Processing system 902 may be implemented within a single processing device, but may also be distributed across multiple processing devices or sub-systems that cooperate in executing program instructions. Examples of processing system 902 include general purpose central processing units, application specific processors, and logic devices, as well as any other type of processing device, combinations, or variations thereof.

Storage system 903 may comprise any computer readable storage media readable by processing system 902 and capable of storing software 905. Storage system 903 may include volatile and nonvolatile, removable and non-removable media implemented in any method or technology for storage of information, such as computer readable instructions, data structures, program modules, or other data. Examples of storage media include random access memory, read only memory, magnetic disks, optical disks, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 903 may also include computer readable communication media over which at least some of software 905 may be communicated internally or externally. Storage system 903 may be implemented as a single storage device, but may also be implemented across multiple storage devices or sub-systems co-located or distributed relative to each other. Storage system 903 may comprise additional elements, such as a controller, capable of communicating with processing system 902 or possibly other systems.

Software 905 may be implemented in program instructions and among other functions may, when executed by processing system 902, direct processing system 902 to operate as described with respect to the various operational scenarios, sequences, and processes illustrated herein. For example, software 905 may include program instructions for directing the system to perform the processes described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 905 may include additional processes, programs, or components, such as operating system software, virtual machine software, or application software. Software 905 may also comprise firmware or some other form of machine-readable processing instructions executable by processing system 902.

In general, software 905 may, when loaded into processing system 902 and executed, transform a suitable apparatus, system, or device (of which computing system 901 is representative) overall from a general-purpose computing system into a special-purpose computing system. Indeed, encoding software on storage system 903 may transform the physical structure of storage system 903. The specific transformation of the physical structure may depend on various factors in different implementations of this description. Examples of such factors may include, but are not limited to, the technology used to implement the storage media of storage system 903 and whether the computer-storage media are characterized as primary or secondary storage, as well as other factors.

For example, if the computer readable storage media are implemented as semiconductor-based memory, software 905 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 907 may include communication connections and devices that allow for communication with other computing systems (not shown) over communication networks (not shown). Examples of connections and devices that together allow for inter-system communication may include network interface cards, antennas, power amplifiers, RF circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

User interface system 909 may include a keyboard, a mouse, a voice input device, a touch input device for receiving a touch gesture from a user, a motion input device for detecting non-touch gestures and other motions by a user, and other comparable input devices and associated processing elements capable of receiving user input from a user. Output devices such as a display, speakers, haptic devices, and other types of output devices may also be included in user interface system 909. In some cases, the input and output devices may be combined in a single device, such as a display capable of displaying images and receiving touch gestures. The aforementioned user input and output devices are well known in the art and need not be discussed at length here. In some cases, the user interface system 909 may be omitted when the computing system 901 is implemented as one or more server computers such as, for example, blade servers, rack servers, or any other type of computing server system (or collection thereof).

User interface system 909 may also include associated user interface software executable by processing system 902 in support of the various user input and output devices discussed above. Separately or in conjunction with each other and other hardware and software elements, the user interface software and user interface devices may support a graphical user interface, a natural user interface, or any other type of user interface, in which a user interface to a productivity application may be presented.

Communication between computing system 901 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses, computing backplanes, or any other type of network, combination of network, or variation thereof. The aforementioned communication networks and protocols are well known and need not be discussed at length here. In any of the aforementioned examples in which data, content, or any other type of information is exchanged, the exchange of information may occur in accordance with any of a variety of well-known data transfer protocols.

The functional block diagrams, operational scenarios and sequences, and flow diagrams provided in the Figures are representative of exemplary systems, environments, and methodologies for performing novel aspects of the disclosure. While, for purposes of simplicity of explanation, methods included herein may be in the form of a functional diagram, operational scenario or sequence, or flow diagram, and may be described as a series of acts, it is to be understood and appreciated that the methods are not limited by the order of acts, as some acts may, in accordance therewith, occur in a different order and/or concurrently with other acts from that shown and described herein. For example, those skilled in the art will understand and appreciate that a method could alternatively be represented as a series of interrelated states or events, such as in a state diagram. Moreover, not all acts illustrated in a methodology may be required for a novel implementation.

The descriptions and figures included herein depict specific implementations to teach those skilled in the art how to make and use the best option. For the purpose of teaching inventive principles, some conventional aspects have been simplified or omitted. Those skilled in the art will appreciate variations from these implementations that fall within the scope of the invention. Those skilled in the art will also appreciate that the features described above can be combined in various ways to form multiple implementations. As a result, the invention is not limited to the specific implementations described above, but only by the claims and their equivalents.

What is claimed is:

1. An article of footwear comprising:
a sole having a plurality of pressure chambers;
a plurality of pressure sensors that take pressure readings from the plurality of pressure chambers;
a motion sensor that senses motion of the article of footwear;
a microcontroller operatively coupled with the plurality of pressure sensors and the motion sensor and configured to:
detect when a subject enters a standing state from the motion of the article of footwear; and
in response to detecting the standing state, determine a weight applied by the subject against the sole from the pressure readings,
wherein the plurality of pressure chambers are disposed to support the weight applied by the subject against the sole in the standing state.

2. The article of footwear of claim 1, wherein the plurality of pressure chambers comprise hydraulic chambers molded into at least four quadrants of the sole, and wherein the pressure readings comprise hydraulic pressure readings.

3. The article of footwear of claim 1, wherein to determine the weight applied by the subject against the sole, the microcontroller is configured to:
sample the pressure readings from the plurality of pressure sensors;
convert the pressure readings to corresponding weight measurements; and
sum the weight measurements to determine the weight applied by the subject against the sole.

4. The article of footwear of claim 3, wherein to convert the pressure readings to corresponding weight measurements, the microcontroller applies quadratic equations with different predetermined coefficients for each of the plurality of pressure chambers.

5. The article of footwear of claim 4, wherein to convert the pressure readings to corresponding weight measurements, the microcontroller is further configured to adjust the weight measurements based on ambient sensor readings.

6. The article of footwear of claim 1, further comprising:
a wireless transceiver configured to communicate with at least one of an opposing article of footwear or an auxiliary electronic device.

7. The article of footwear of claim 6, wherein the microcontroller is further configured to:
receive a weight applied by the subject against a sole of the opposing article of footwear; and
sum the weight applied by the subject against the sole of the opposing article with the weight applied by the subject against the sole of the article of footwear to determine a total weight of the subject.

8. The article of footwear of claim 7, wherein the wireless transceiver is configured to communicate the total weight of the subject to at least the auxiliary electronic device.

9. The article of footwear of claim 1, wherein the microcontroller is further configured to sample the pressure readings from one or more of the plurality of pressure sensors for activity monitoring.

10. The article of footwear of claim 9, wherein the activity monitoring comprises monitoring one or more of running or walking gait, foot strike, left-right balance, or step counting.

11. The article of footwear of claim 1, wherein at least a portion of the microcontroller functionality is molded or embedded in the sole.

12. The article of footwear of claim 1, wherein at least one of the plurality of pressure sensors or the motion sensor is molded or embedded in the sole.

13. An apparatus comprising:
one or more computer readable storage media storing program instructions that, when executed by one or more processing systems of an article of footwear, direct the one or more processing systems to:
monitor a motion sensor that senses motion of the article of footwear;
detect when a subject enters a standing state from the motion of the article of footwear;
in response to detecting the standing state, sample a plurality of pressure sensors of the article of footwear that take pressure readings from a plurality of pressure chambers molded or embedded in a sole of the article of footwear; and
determine a weight applied by the subject against the sole from the pressure readings, wherein the plurality of pressure chambers are disposed to support the weight applied by the subject against the sole in the standing state.

14. The apparatus of claim 13, wherein to determine the weight applied by the subject against the sole, the program instructions, when executed by the one or more processing systems of the article of footwear, direct the one or more processing systems to:
sample the pressure readings from the plurality of pressure sensors;
convert the pressure readings to corresponding weight measurements; and
sum the weight measurements to determine the weight applied by the subject against the sole.

15. The apparatus of claim 14, wherein to convert the pressure readings to corresponding weight measurements, the program instructions, when executed by the one or more processing systems of the article of footwear, direct the one or more processing systems to:
sample readings from one or more ambient sensors of the article of footwear; and
adjust the weight measurements based on the readings.

16. The apparatus of claim 13, wherein the program instructions, when executed by the one or more processing systems of the article of footwear, further direct the one or more processing systems to:
communicate with at least one opposing article of footwear to obtain a weight applied by the subject against a sole of the opposing article of footwear during the standing state; and
sum the weight applied by the subject against the sole of the opposing article with the weight applied by the subject against the sole of the article of footwear to determine a total weight of the subject.

17. The apparatus of claim 16, wherein the program instructions, when executed by the one or more processing systems of the article of footwear, further direct the one or more processing systems to:
direct a wireless transceiver of the article of footwear to communicate the total weight of the subject to an auxiliary electronic device.

18. The apparatus of claim 13, wherein the program instructions, when executed by the one or more processing systems of the article of footwear, further direct the one or more processing systems to:
sample the pressure readings from one or more of the plurality of pressure sensors; and
detect an activity of the subject based on the pressure readings.

19. The apparatus of claim 18, wherein the activity includes one or more of running or walking gait, foot strike, left-right balance, or step counting.

20. A system comprising:
a plurality of articles of footwear that communicate with each other, each article of footwear including:
a sole having a plurality of pressure chambers to support a weight applied by a subject against the sole while in a standing state;
a plurality of pressure sensors that take pressure readings from the plurality of pressure chambers;
a motion sensor that senses motion of the article of footwear; and
a microcontroller operatively coupled with the plurality of pressure sensors and the motion sensor and configured to detect when the subject enters the standing state from the motion of the article of footwear, and responsively determine a weight applied by the subject against the sole from the pressure readings.
